(19) United States
(12) Patent Application Publication
(10) Pub. No.: US 2013/0130303 A1
(43) Pub. Date: May 23, 2013

(54) METHODS FOR SCREENING MICROBIAL REMEDIATION AGENTS

(71) Applicant: Saint Louis University, a non-profit organization, St. Louis, MO (US)

(72) Inventors: Anupma Dixit, St. Louis, MO (US); Brunda Tumala, Chesterfield, MO (US)

(73) Assignee: Saint Louis University, a non-profit organization, St. Louis, MO (US)

(21) Appl. No.: 13/683,927

(22) Filed: Nov. 21, 2012

Related U.S. Application Data
(60) Provisional application No. 61/563,390, filed on Nov. 23, 2011.
(60) Provisional application No. 61/563,375, filed on Nov. 23, 2011.

(51) Int. Cl. C12Q 1/18

(52) U.S. Cl. CPC C12Q 1/18
USPC 435/32

(57) ABSTRACT

Disclosed are methods for determining the efficacy of antimicrobial agents used in the treatment of building materials after microbial contamination has occurred, for the purpose of killing existing microbial growth and reducing or inhibiting recurrent or subsequent microbial growth. The disclosed methods may be used to determine microbial growth at time points subsequent to antimicrobial treatment of the material surface. The disclosed invention also measures visible microbial growth in a semi-quantitatively analysis. In addition, the Inventors disclosure a method with reduced variability and a more accurate assessment of antimicrobial efficacy.

### CROSS REFERENCE TO RELATED APPLICATIONS

This application claims priority to provisional application 61/563,390, filed Nov. 23, 2011, and provisional application 61/563,375 filed Nov. 23, 2011, both of which are hereby incorporated by reference in its entirety.

### GOVERNMENT SUPPORT CLAUSE

This work was supported by HUD grant MOLHH0167-08 and MOLHH0195-09. The U.S. Government has certain rights in this invention.

### FIELD OF THE INVENTION

The invention relates to methods and compositions for testing the antimicrobial activities of known and experimental antimicrobial formulations on building materials for the purpose of killing existing microbial growth and reducing or inhibiting recurrent or subsequent microbial growth.

### BACKGROUND

Damp buildings are associated with a spectrum of adverse health outcomes in sensitive individuals (Institute of Medicine. (2004) The National Academies Press, Washington, D.C., and World Health Organization (2009) ISBN 798 92 890 4168 3, Publications: WHO Regional Office for Europe, Copenhagen, Denmark). Fungal (mold) growth on indoor surfaces following moisture incursion represents a potential health hazard for the building occupants. A US cost estimate for asthma attributable to indoor dampness and mold exposure alone was $3.5 billion (Mudarri, and Fisk (2007) Indoor Air. 17:226-235). Decontamination of impermeable surfaces with disinfectants is an accepted practice but the treatment of moldy or moist semiporous building materials with fungicides is not promoted. Many mold remediation guidelines agree that moldy or permeable and semipermeable materials wet for >24 hours must be discarded (Cole and Foarde (1999) Biocides and Antimicrobial Agents. In: J. Macher (ed.). Bioaerosols: Assessment and Control. American Conference of Governmental and Industrial Hygienists, Cincinnati, Ohio). Nonetheless, there is a growing recognition that sometimes it is not done or there are situations where discarding is not always feasible or desirable (Krause et al., (2006) J. Occup. Environ. Hyg. 3:435-441; Menetrez, et al., (2008) J. Occup. Environ. Hyg. 5:63-66; Price and Ahearn, (1999) Curr. Microbiol. 39: 21-26), such as when complete mold removal is not feasible (Menetrez, et al., (2007) J. Occup. Environ. Hyg. 5:63-66), or if encapsulation is desired post-cleaning (Krause at al., (2006) J. Occup, Environ. Hyg. 3:435-441).

In a recent survey of commercial mold remediation practices, a significant proportion of the survey participants acknowledged either using or suggesting antimicrobial product application during the remediation (Dixit, A. (2008). A national survey of professional mold remediation practices. National Healthy Homes Conference), Antimicrobial products are used separately or in combination with other remedial measures in mold remediation and water damage restoration situations though supportive data has been lacking. To date, only a small number of studies have examined the effectiveness of common surface treatment methods such as cleaning of moldy gypsum wallboard by dry brushing, wiping, and/or treatment with chemicals (Krause et al., (2006) J. Occup. Environ. Hyg. 3:435-441. Menetrez, at al., (2007) J. Occup. Environ. Hyg. 5:63-66; Price and Ahearn, (1999) Curr. Microbiol. 39: 21-26). But, in particular, information regarding the efficacy of industrial-strength high airflow vacuum cleaning in removal of the existing growth of fungi on moldy building surfaces, with or without subsequent treatment with antimicrobial agents is absent. The Inventors have discovered a method of determining antimicrobial effectiveness in conjunction with the removal of gross fungal contamination from moldy material surfaces using an industrial-strength High Efficiency Particulate Arrestor (HEPA) filter-fitted vacuum cleaner.

Current methods of testing do not take into account the physical differences between building materials or environmental requirements of a broad range of microbes. Building materials differ with respect to composition and porosity which directly influences the rate of absorption, diffusion, and/or leaching of antimicrobials both during and after treatment. Building materials also provide a range of growth environments for microbes. It is not possible to simulate all of these factors with existing methods. The disclosed method can be used to test formulations against bacteria or fungi differing in morphology, nutrition, as well as substrate temperature and moisture requirements. The use of building materials as substrates allows for the determination of antimicrobial activities of experimental formulations on a wide range of microbes with varied nutritional and environmental requirements, and is not limited to those that grow only on filter paper under high humidity. In addition, existing methods determine growth by solely through visual observation, and require at least several days for mold to grow on a substrate before it can be assessed. The Inventors discovered that even if there is no visible fungal growth on a substrate, viable fungal elements may remain which can be detected only by extraction of the substrate and subsequent culture to determine colony counts. The disclosed methods may be used to determine microbial growth at time points subsequent to antimicrobial treatment of the material surface and exposure to microorganisms. In addition, the disclosed invention also measures visible microbial growth semi-quantitatively instead of a subjective visual estimate approach. For example, by determining the percent of the substrate area covered with fungi using a quantitative grid, data obtained can be then converted to scores based on a 0-5 rating system. The result is more accurate and less subjective than estimates of growth used previously.

The inventors have discovered a method by which to test a broad range of antimicrobials on building materials, finishing, and furnishing materials under a broad range of environmental conditions. In addition, they have discovered a method that has reduced variability and arrives at a more accurate assessment of antimicrobial efficacy.

### SUMMARY OF THE INVENTION

A method of determining the effectiveness of an antimicrobial agent in reducing or inhibiting recurrent microbial growth, the method comprising:
a) obtaining a substrate with substantial microbial growth or gross contamination,
b) removing the substantial microbial growth or gross contamination from the substrate by mechanically cleaning the substrate,
c) treating the substrate with the antimicrobial agent,
d) incubating the substrate for one or more periods of time at a temperature and relative humidity that will allow growth of the microbe,
e) determining viable colony forming units by extracting viable microbes from the substrate and plating the extract on nutritive media, then incubating in an environment that allows growth of the microbes and,
f) comparing the viable colony forming units obtained from the antimicrobial treated substrates, to viable colony forming units obtained from substrates not treated with an antimicrobial or treated with different antimicrobials, and subjected to similar conditions.

A method of determining the effectiveness of an antimicrobial agent in reducing or inhibiting microbial growth on a substantially contaminated substrate, the method comprising:
a) obtaining a substrate with substantial microbial growth or gross contamination,
b) mechanically cleaning the substrate to remove with substantial microbial growth or gross contamination,
c) treating a substrate with the antimicrobial agent,
d) incubating the substrate for one or more periods of time to determine efficacy of the antimicrobial, at a temperature and relative humidity that will allow growth of the microbe,
e) assessing substrate surface growth,
f) determining viable colony forming units by extracting viable microbes from the substrate and plating the extract on appropriate nutritive media and under appropriate environmental conditions that would allow growth of the microbe and,
g) comparing the viable colony forming units and the assessed substrate surface growth, obtained from the antimicrobial treated substrates to viable colony forming units and assessed substrate surface growth obtained from substrates not treated with an antimicrobial, or to viable colony forming units obtained from substrates treated with different antimicrobials and subjected to similar conditions.

### DESCRIPTION OF THE FIGURES

FIG. 1 illustrates a testing protocol that utilizes building materials as substrates and residual viability testing, and/or substrate surface growth.

FIG. 2 illustrates average (±SD) percent (%) log reduction in residual viability of fungi on building substrates 5 weeks after HEPA vacuum cleaning and subsequent coating treatment (n=5).

FIG. 2.1 illustrates one alternative method of expressing the data shown in FIG. 2, where the average log reduction or geometric mean reduction±SD (geometric standard deviation) in fungal viability after each antimicrobial agent is compared to a water control.

FIG. 3 illustrates average percent (±SD) visible regrowth of A. alternaia on gypsum wallboard after HEPA vacuum cleaning and treatment with fungicidal products (n=3). dH2O=Distilled Water Control, DOT=Disodium Octaborate Tetrahydrate, CP1=Chlorine Product 1, CP2=Chlorine Product 2, CP3=Chlorine Product 3, HP1=Hydrogen Peroxide Product 1, HP2=Hydrogen Peroxide Product 2, PP1=Phenol Product 1, PP2=Phenol Product 2, Quat 1=Quaternary Ammonium Compound 1, Quat 2=Quaternary Ammonium Compound 2, Quat 3—Quaternary Ammonium Compound 3.

FIG. 4 illustrates average percent (±SD) visible regrowth of C. globosum on gypsum wallboard after HEPA vacuum cleaning and treatment with fungicidal products (n=3). dH2O=Distilled Water Control, DOT=Disodium Octaborate Tetrahydrate, CP1=Chlorine Product 1, CP2=Chlorine Product 2, CP3=Chlorine Product 3, HP1=Hydrogen Peroxide Product 1, HP2=Hydrogen Peroxide Product 2, PP1=Phenol Product 1, PP2=Phenol Product 2, Quat 1=Quaternary Ammonium Compound 1, Quat 2=Quaternary Ammonium Compound 2, Quat 3—Quaternary Ammonium Compound 3.

FIG. 5 illustrates average (±SD) percent (%) log reduction in residual viability of fungi on building substrates five weeks after HEPA vacuum cleaning and subsequent treatment with chemicals (n=3). Log reduction values were derived by comparison with negative control treatment. Gypsum wallboard was used as carrier for A. alternata, C. globosum, and C. sphaerospermum, and Oriented Strand Board (OSB) as a substrate for P. brevicompactum. dH2O=Distilled Water Control, DOT=Disodium Octaborate Tetrahydrate, CP1=Chlorine Product 1, CP2=Chlorine Product 2, CP3=Chlorine Product 3, HP1=Hydrogen Peroxide Product 1, HP2=Hydrogen Peroxide Product 2, PP1=Phenol Product 1, PP2=Phenol Product 2, Quat 1=Quaternary Ammonium Compound 1, Quat 2=Quaternary Ammonium Compound 2, Quat 3—Quaternary Ammonium Compound 3.

FIG. 5.1 illustrates one alternative method of expressing the data shown in FIG. 5, where the average log reduction or geometric mean reduction±SD (geometric standard deviation) in fungal viability after antimicrobial agent is compared to a water control.

FIG. 6 illustrates average (±SD) percent (%) log reduction in residual viability of fungi on building substrates five weeks after HEPA vacuum cleaning and subsequent treatment with green chemicals (n=5). Log reduction values were derived by comparison with negative control treatment. Gypsum wallboard was used as carrier for A. versicolor, and S. chartarum, and Plywood as a substrate for P. brevicompactum.

FIG. 6.1 illustrates one alternative method expressing the data shown in FIG. 6, where the average log reduction or geometric mean reduction±SD (geometric standard deviation) in fungal viability after antimicrobial agent is compared to water control.

### DETAILED DESCRIPTION

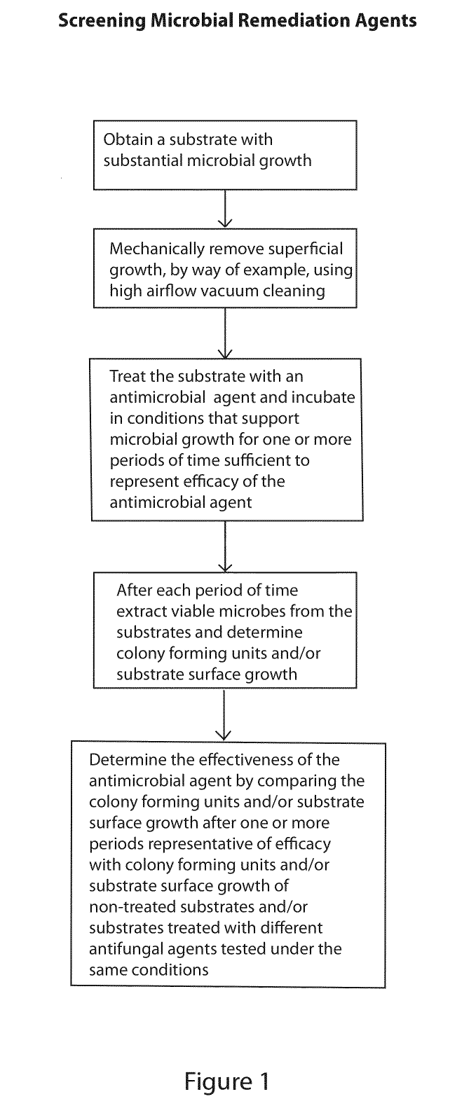

Disclosed herein are methods for determining the efficacy of antimicrobial agents used in the treatment or coating of building materials after microbial contamination has occurred, for the purpose of killing existing microbial growth and preventing recurrent or subsequent microbial growth. As illustrated by the non-limiting example in FIG. 1, substrates containing substantial microbial growth are obtained, cleaned of gross contamination by mechanical means, treated with antimicrobial agents, and assessed for colony forming units at one or more periods of time representative of antimicrobial efficacy. Optionally, or additionally, at the same or different time periods microbial substrate surface growth may be assessed. Colony forming units and substrate surface growth assessments may be compared to similarly treated control substrates that were not treated, treated with a control solution, by way of example water, or treated with different antimicrobial agents.

The disclosed methods utilize building materials that are representative of those used in the field. The term building material is meant to include any and all materials that may be used in building construction, including materials used in building furnishing, and building finishing. The disclosed methods may be used with any material for which it is desirable to treat that material with antimicrobials. The samples, coupons, or carriers of building materials disclosed in the test methods and examples are referred to herein collectively as substrates. Any material used in building construction or building furnishing may be used as a substrate. Non-limiting examples of building materials include: nonporous materials, including hard surface materials; semiporous materials including drywall, engineered wood, wood, particle board, oriented strand board, and framing lumber; flooring materials, including carpet, carpet padding, wood, and laminate; as well as porous materials, including insulation, textile, and synthetic material furnishings.

Substrates may be obtained in a contaminated state from the field or microbial growth may be cultivated on substrates in the laboratory. Microbial growth may be comprised of one or more microorganisms. Once a substrate with substantial microbial growth is obtained, gross contamination is physically removed through mechanical means. In one example, a high airflow vacuum cleaner is used. A HEPA vacuum is preferred due to its high strength and filtration system designed to protect the operator. Substrates may then be treated with the particular antimicrobial agent to be tested. Any method of applying the antimicrobial treatment may be utilized. A preferred method is that method which is recommended by the manufacturer for the particular antimicrobial agent and the substrate. Substrates may be cut to a convenient size to assist in handling, or for assessments based on surface area at any convenient time prior to analysis. By way of example, a substantially contaminated dry wall, engineered wood, or carpeting may be obtained and cut into substrates of 2×2×0.5 inches, cleaned of gross contamination with a HEPA vacuum, and then sprayed, immersed or painted with an antimicrobial according to the manufacturer's recommendations. The substrate may then be incubated in sterile conditions at a moderate temperature and relative humidity (RH), for example 25° C. and 65% RH, or a temperature and RH known to be supportive or optimum for the test microbes. The substrates may then be incubated for one or more periods of time representative of antimicrobial efficacy. By way of example, the substrates may be incubated for periods of time of about 24 hours and/or about 5 weeks, or periods of time representative of short term efficacy and/or long term efficacy of the antimicrobial agent. After each time period, viable microbes may be recovered and cultured in nutrient media to determine colony forming units.

Colony forming units may be determined by any number of methods known in the art. By way of example, substrates may be individually placed inside sterile bags or containers and washed with a known volume of a sterile aqueous solution, for example, phosphate buffered saline, and microbes extracted for several minutes or hours, preferably with agitation. After appropriate dilution, resultant extracts may be plated on nutritive media, for example agar plates and the number of colonies determined after an appropriate period of time, for example 1 day to 10 days, depending on the test microbe. By way of general example, bacteria may typically require about 24 hours whereas fungi may typically require 2 days or longer before colonies may be detected visually without the aid of a microscope. The number of colony forming units may be expressed as microbes per milliliter in the original extract solution and/or may be used to determine the number of colony forming units per unit area of the substrate. A greater reduction in colony forming units per milliliter or per unit area will indicate greater efficacy at that particular time point.

Microbial growth on the substrate surface may also be assessed nondestructively. By way of a preferred example, the area of visible microbial growth on the substrate surface may be measured by placing a grid over the culture dish and counting the number of squares occupied by microbial growth on the surface of the substrate. The area of growth on the substrate surface may be expressed as a percentage of the total area. Alternatively, once the area of microbial growth has been measured, the degree of microbial growth on the surface of the substrate may be scored using the non-limiting criteria are set forth in Table 1

TABLE 1
Rating Scheme for Visible Growth on Substrate Surfaces

| Visible Percent | |
|---|---|
| Growth | Rating |
| 75-100% | 5 |
| 50-74% | 4 |
| 30-49% | 3 |
| 15-29% | 2 |
| 5-14% | 1 |
| <5% Traces of | 0 |
| Growth | |

It is fully apparent that microbes grow on building materials and building furnishings in moist environments in the field. Therefore, another embodiment of the invention is to measure antimicrobial efficacy in the field using the natural environment to provide test microbes, and/or the culture conditions, including temperature and relative humidity. One such embodiment would obtain contaminated substrates from the field, which would be cleaned of gross contamination and returned to the field after treatment with antimicrobial agents. Colony forming units and substrate surface growth may be assessed at time periods representative of antimicrobial efficacy as illustrated in FIG. 1. In yet another embodiment, microbial growth may be cultivated on substrates in the laboratory. Substrates may then be cleaned of gross contamination, treated with antimicrobials, and then placed in the field for incubation before subsequent assessment of colony forming units and/or substrate surface growth. It is expected that comparisons made in the field will be between substrates incubated under similar or identical test sites. For example, treated substrates and non-treated substrates may be incubated adjacent to one another at the test site or equivalent test sites in the field.

Regardless of the particular embodiment of the invention, inhibition of microbial growth represents antimicrobial activity or efficacy. The results may be expressed in any number of methods known in the art. Methods of comparing treated samples to non-treated samples are well known in the art and are commonly referred to as the use of controls. By way of example, results of a particular antimicrobial treatment, may be compared to substrates, which did not receive antimicrobial treatment, but have been subject to the testing under the same conditions, in particular, the same test microbial number or concentrations of test microbes, as well as the same temperature and RH, and methods of comparing colony forming units, or visible growth on the substrate surface after the same short term and/or long term efficacy time periods. Growth or inhibition may be expressed as percentage of the growth or inhibition of microbes on the non-treated or control substrate. In another example, the relative efficacy of different antimicrobials may be directly compared by testing different antimicrobials using the same conditions, in particular, the same test microbial number or concentrations of test microbes, as well as the same temperature and RH, and methods of comparing colony forming units, or visible growth on the substrate surface after the same short term and/or long term efficacy time periods. Results from any of the described analysis may be expressed through any mathematical transformation including but not limited to log. Any statistical comparisons known in the art may be used to compare quantitative data. Non-limiting examples include t-Test, and ANOVA or General Linear Modeling followed by Post Hoc analysis. The p-value for the acceptable level of statistical significance in mean difference between treatments should be predetermined.

### Test Microbes

Large and diverse groups of microbes are known to colonize building materials that have become moist or water-damaged. Any microbe that may grow or be cultured on building materials, either singly or as a mixture of microbes, and whereby gross contamination by the microbe may be mechanically removed, may be used as a test microbe. Non-limiting examples include bacterial and fungal microbes found at American Type Culture Collection (ATCC) (P.O. Box 1549, Manassas, Va. 20108). By way of example, fungal strains that are known to colonize a variety of moist building materials include: Alternaria alternate (ATCC® 58868™), Aspergillus versicolor (ATCC®16856™); Chaetomium globosum (ATCC® 34507™); Cladosporium sphaerospermum (ATCC® 11293™); Penicillium brevicompactum (ATCC® 9056™); and Stachybotrys chartarum (ATCC® 8541™). Examples of most preferred test microbes include fungi that frequently colonize moist building materials, including gypsum wallboard and wood products including Alternaria alternate, Aspergillus versicolor and other Aspergillus spp., Chaetomium globosum, Cladosporium spp., Penicillium app., Stachybotrys chartarum and Trichoderma app.

Substrates with Substantial Microbial Growth

As illustrated in FIG. 1, the method utilizes a substrate with substantial microbial growth. Substrates with substantial microbial growth are also referred to herein as substrates with substantial contamination. To obtain a substrate with substantial microbial growth, the user may obtain contaminated building materials from the field, or may cultivate equivalent contaminated building materials in the laboratory, Substrates with substantial microbial growth obtained from the field may be collected from any site where building materials or furnishing materials are exposed to moisture and microbes, typical to a construction site in a moist environment. Substrates with substantial microbial growth collected from the field are preferably collected in close proximity to one another in order to reduce variability between the replicates and treatments. Substrates with substantial microbial growth may be cultivated in the laboratory by inoculating a substrate, preferably sterile, with one or more microbes and incubating under temperature and relative humidity which allow the test microbes to grow, Any number of methods may be employed to inoculate substrates with test microbes. It is preferable that they result in an evenly dispersed known number of microbes. Typically this requires adjusting the volume and/or number or concentration of test microbes in the inoculum or suspension and applying a known volume of inoculum evenly to the substrate with a dispersal device. Examples of methods used to evenly disperse a known number of test microbes include pipetting a known volume of a known number of test microbes on to a surface of the substrate, and evenly dispersing the volume with a sterile glass rod across the surface. Evenly dispersing a known number of test microbes reduces variation between the replicates and treatments. In addition, the top surface of each substrate is evenly dispersed in the same manner, further reducing variability between replicates and treatments. In order to be consistent between groups, typically the same or equivalent suspension will be utilized as an inoculum and the volume adjusted according to the size of the substrate or the desired number of test microbes to be applied. Incubation of the substrates may continue until the microbial growth or contamination becomes substantial. Substantial growth generally means that the level of growth is at least extensive enough to be visible with the unaided eye.

### Culture Conditions and Incubation Times

The disclosed invention utilizes techniques generally known to artisans in the field. These techniques are described in detail in numerous laboratory protocols, one of which is entitled, A Photographic Alias for the Microbiology Laboratory, by Leboffe and Pierce, (3^{rd} ed., 2005, Morton Publishing Co. Engelwood, Calif.), hereby incorporated by reference in its entirety. One of ordinary skill in the art may determine culture conditions for a given test microbe that are supportive or optimal for the purposes of practicing the invention without undue experimentation. The selection of test conditions may be based on information available regarding the original source of isolation, substrate preference, and biodeteriogenic activity from American Type Culture Collection (ATCC), and/or any other source, of information available including the scientific literature.

In at least one embodiment it is envisioned that the test materials may be obtained in the field and/or incubated in the field, Contaminated materials may be obtained at or incubated at any site where building materials or furnishing materials are exposed to moisture and microbes before, during, and after construction. These are typically construction sites, or any site where a building may be built or potentially may be built. Non-limiting examples include existing structures in flood zone or low lying areas and buildings subjected/prone to moisture incursion through onetime, chronic or catastrophic water events. It is well known that conditions at these sites will support microbial, including fungal growth.

Similarly, one of ordinary skill in the art may also determine incubation times that are representative of short term and long term efficacy of antimicrobial agents by taking into account physicochemical properties of the antimicrobial and/or the particular building material being tested. Incubation times representing short or long term efficacy are also referred to herein as incubation times, incubation periods, or, as in the examples, referred to in reference to time periods where substrates are in contact with, or exposure to microbes. Physicochemical properties of the antimicrobial, including active half-life, solubility, and leaching times from particular materials may be based on information available from the manufacturer and/or the scientific literature.

It is not necessary that the test conditions or incubation times be optimal to practice the invention. It is only necessary that the test conditions and incubation times support growth and remain consistent between treated and non-treated substrates, or between treatment groups being subject to comparison.

### Substrates

Substrates include any and all materials used in building, constructing, finishing, and furnishing buildings. Non-limiting examples of building materials include: nonporous materials, including hard surface materials; semiporous materials including drywall, engineered wood, wood, particle board, oriented strand board, and framing lumber; flooring materials, including carpet, carpet padding, wood, and laminate; as well as porous materials, including insulation, textile, and synthetic material furnishings. The term substrate as used herein is meant to include the coupons and carriers referred to in the examples.

### Antimicrobials

Antimicrobials or antimicrobial agents include any natural occurring or manufactured compound that possess microbial killing or growth inhibiting properties, including any compound with fungistatic, fungicidal, bacteriostatic or bactericidal activity that may be applied to a building or furnishing material. The term antimicrobials as used herein is meant to include the antimicrobial coatings, or chemicals referred to in the examples. Non-limiting preferred examples of antimicrobials are listed in Tables 2, 3, and 4.

### EXAMPLES

### Materials and Methods

These methods and materials were used in each of the following examples unless otherwise specified.

### Antimicrobial Agents:

Coatings: antimicrobial coatings Tested are listed in Table 2. Coatings were selected based on active ingredients, not for product-label verification.

TABLE 2
Antimicrobial coatings

| Antimicrobial | |
|---|---|
| coating | |
| products | Active ingredients* |
| Coating 1 | Titanium dioxide[<25%], 2-tetrachloroisophthalonitrile |
| | [0.49%] |
| Coating 2 | Barium compound (15%) with propynyl butyl |
| | carbamate (17%) |
| Coating 3 | Barium compound [5-10%], titanium dioxide [1-5%], |
| | zinc oxide [1-5%], amorphous silica [1-5%], and |
| | propylene glycol [0.1-1%] |
| Coating 4 | 3-Iodo-2 propynyl butyl carbamate (1%) |
| Coating 5 | Organosilane |

*Based on the information available on product label, material safety data sheet, or technical information.

Fungicides: Ten EPA-approved commercial remediation formulations and household bleach were chosen for efficacy testing, Mold remediation formulations were purchased from manufacturer or distributor, and household bleach was obtained from a local grocery store. The chemical class, active ingredients, and recommended concentration for use are presented in Table. 3. Each test chemical represented formulation with one of the highest concentrations of the active ingredients in its chemical class or category.

TABLE 3
Fungicides

| | | Ready To Use |
|---|---|---|
| | | (RTU)/ |
| | | Recommended |
| Chemical product | Active ingredients* | dilution |
| DOT | Disodium Octaborate | 15% w/v |
| | Tetrahydrate | |
| Chlorine Product 1 | Sodium Hypochlorite (5.7%) | 1:10 |
| (CP1) | | |
| Chlorine Product 2 | Chlorine dioxide (0.72%), | 1:4 |
| (CP2) | Didecyl dimethyl ammonium | |
| | chloride (0.4%) | |
| Chlorine Product 3 | Stabilized Chlorine dioxide | RTU |
| (CP3) | (<1%), Alkyl Dimethyl Benzyl | |
| | Ammonium Chloride (<1%), | |
| | Alkyl Dimethyl ethylbenzyl | |
| | Ammonium Chloride (<1%) | |
| Hydrogen Peroxide | Hydrogen Peroxide (<10%) | RTU |
| Product 1 (HP1) | | |
| Hydrogen Peroxide | 3-Propyl dimethyl octadecyl | 1:16 |
| Product 2 (HP2) | ammonium chloride (<1%), | |
| | Hydrogen Peroxide (<5.5%), | |
| | Ethylene glycol monobutyl ether | |
| | (<6%), | |
| Phenol Product 1 | Phenyl Phenol (0.22%), | RTU |
| (PP1) | Dimethyl benzyl ammonium | |
| | chloride monohydrate (0.74%) | |
| Phenol Product 2 | Phenol (1.56%) and Sodium | RTU |
| (PP2) | phenate (0.06%) | |
| Quaternary | Octyl decyl dimethyl ammonium | RTU |
| Ammonium | chloride (0.025%), Dioctyl | |
| Compound 1 | dimethyl ammonium chloride | |
| (Quat 1) | (0.01%), Didecyl dimethyl | |
| | ammonium chloride (0.0155), | |
| | Alkyl dimethyl benzyl | |
| | ammonium chloride (0.034%) | |
| Quaternary | Dialkyl Dimethyl Ammonium | 1:64 |
| Ammonium | Chloride (3.3%), Alkyl Dimethyl | |
| Compound 2 | Benzyl Ammonium Chloride | |
| (Quat 2) | (2.2%) | |
| Quaternary | 1-Dimethyl Benzyl Ammonium | 1:64 |
| Ammonium | Chloride (<2.25%), 2-Dimethyl | |
| Compound 3 | Ethylbenzyl Ammonium | |
| (Quat 3) | Chloride (<2.25%) | |

*Based on information available on product label, Material Safety Data Sheet, or Technical Data Sheet.

Green Antimicrobial Products: Seven mold remediation formulations currently marketed as “green” were chosen for fungicidal efficacy testing. In addition, 30% ethanol made by diluting denatured ethyl alcohol (Fisher Chemical A407-4) in sterile distilled water was tested. Active ingredients and recommended concentration for use are presented in Table 4.

TABLE 4
Green Antimicrobial Formulations

| | | Ready To |
|---|---|---|
| | | Use (RTU)/ |
| | | Recommended |
| ID # | Green product | Dilution |
| 1 | Green Product 1 (0.23% Thymol) | RTU |
| 2 | Green Product 2 (Pure Australian Tea tree oil and | RTU |
| | unnamed essential oils) | |
| 3 | Green Product 3 (0.95% Sodium carbonate (soda | RTU |
| | ash) with patented blend of Inorganic compounds) | |
| 4 | Green Product 4 (Water and unnamed plant based | 1:8 |
| | product) | |
| 5 | Green Product 5 (30% ethanol) | 30% Ethanol |
| 6 | Green Product 7 (Clove bud oil) | 1:25 |
| 7 | Green Product 6 (Natural blend of enzymes, | RTU |
| | fermented byproducts of sugarcane and | |
| | vegetables & citric acid) | |
| 8 | Green Product 8 (blend of clove flower bud, | 1:15 |
| | lemon rind, cinnamon bark, eucalyptus leaf, | |
| | rosemary leaf) | |

Substrates:

Building materials for use as experimental substrates included dry gypsum wallboard (SHEETROCK BRAND—Sound Deadening Board#0II8I1099II00065IIIII2—Gypsum Panel Label: WB2248-8/4-2000, Manufacturer: United States Gypsum Co., 125 S. Franklin St., Chicago, Ill. 60606-4678), engineered wood (Plywood Sheathing (3-Ply Rtd Sheathing, MFG Model #132411), and Oriented Strand Board (OSB sheathing, Model #LBR12212) purchased from a local hardware store.

Experimental Fungi:

Test fungi were Alternaria alternate (ATCC® 58868™); Aspergillus versicolor (ATCC® 16856™); Chaetomium globosum (ATCC® 34507™); Cladosporium sphaerospermum (ATCC® 11293™): Penicillium brevicompactum (ATCC® 9056™); and Stachybotrys chartarum (ATCC® 18541™) These fast growing strains exhibited profuse growth on building substrates under experimental conditions (25° C. and 65% RH).

HEPA vacuum cleaning of substrates with substantial contamination (fungal) materials and follow-up treatment with antimicrobial products. The effectiveness of commercial antimicrobial products was tested when applied after removing superficial fungal growth from gypsum dry wallboard and plywood carriers.

Removal of Gross Contamination: The Efficiency of HEPA vacuum cleaning of substantial contamination (fungal) on substrates was evaluated by determining fungal viabilities pre- and post-cleaning. Substrates of 30 mm×30 mm with mature fungal growth (five to six weeks) were mechanically cleaned using a High Efficiency Particulate Arrestor (HEPA) filter equipped vacuum cleaner (Dustless Technologies, Inc. (Airflow: 116.6 cubic feet meter-1)).

Cleaned substrates were subsequently treated with antimicrobial coatings by coating all the sides, including bottom, with a sterile glass spreader. Substrates were passively air-dried in between coatings inside a biosafety cabinet (Level II). However, remediation chemical products (Table 3) and green antimicrobial agents (Table 4) were spray-treated with a ready-to-use or manufacturer-recommended dilution of the formulation. Substrate were sprayed ten times with antimicrobial formulations from a spray bottle (total volume sprayed per carrier sample=1 mL) to completely cover the front surface and four sides covered with growth. Blank substrates without any treatment in case of coatings or treated with sterile distilled water in case of fungicidal chemicals served as the negative controls. Three to five replicates for each treatment and control were used. Antimicrobial treated substrates were incubated at 25° C. and 65% RH and observed for signs of visible fungal growth. In case of antimicrobial coatings, no growth of any test fungi was observed on carrier surface post-cleaning and chemical-treatments. Further, due to the discoloration and residual staining of the substrate after removing growth of A. versicolor, P. brevicompactum and S. chartarum against which green chemicals were evaluated only residual fungal viabilities are reported since growth on the substrate could not be assessed visually. Conversely, visual evaluation (described below) of the recurrence of the growth of A. alternata and C. globosum was possible (only 2 of the 4 species used in fungicide testing) and it continued for 5 weeks. Carrier coupons were extracted to determine residual viabilities following this incubation period.

Visible fungal growth estimation on substrate surface. The non-destructive and semi-quantitative estimation of fungal growth was undertaken by visual evaluation of the substrate surface. Growth was evaluated visually using a transparent plastic 36 square-grid (30 mm×30 mm) that was placed on covered Petri-dish superimposing the substrate to assess carrier surface area covered with vegetative and sporulating fungal mycelium. Results were computed as average percent visible growth indicative of coupon surface area (i.e. no. of squares out of total 36) covered with fungal hyphae.

Recovery of Fungi and Determination of Residual Viability.

After 5 weeks of incubation, substrates were individually placed inside the sterile stomacher bag (Fisher Scientific, Prod#01-002-54) and extracted in 20 mL of the Phosphate Buffered Saline (PBS, pH 7.2) using a stomacher lab blender (Seward lab system, Stomacher® 80) operated for 120 seconds at normal speed. Resultant extracts were plated (100 μL) on nutritive media after appropriate dilution to achieve countable viable colony forming units (CFU's). Viable colonies were counted starting day 3 and followed up to 10 days. The log reductions in fungal viability were calculated based on viable colony forming units (CFU) mL-1 on untreated carrier or negative control. The absence of viable CFU's on antimicrobial treated substrates represented 100% reduction in the viability.

Data Analysis.

Due to the semi-qualitative nature of the outcome of visible growth, statistical analysis was not applicable. When the quantitative outcome was residual viability, colony-forming unit data was normalized by log transformation. A log reduction of 1.0 in average CFU's mL-1 compared to control was used as the cutoff value for the efficiency determination (Urban et al., (2011) J. App. Microbiol. 110:676-687; Zhu et al., (2007) Eye & Contact Lens. 33: 278-283). Average log reduction of 1.0 in viability represented acceptable effect whereas reductions of <1.0 signified negligible difference from the control.

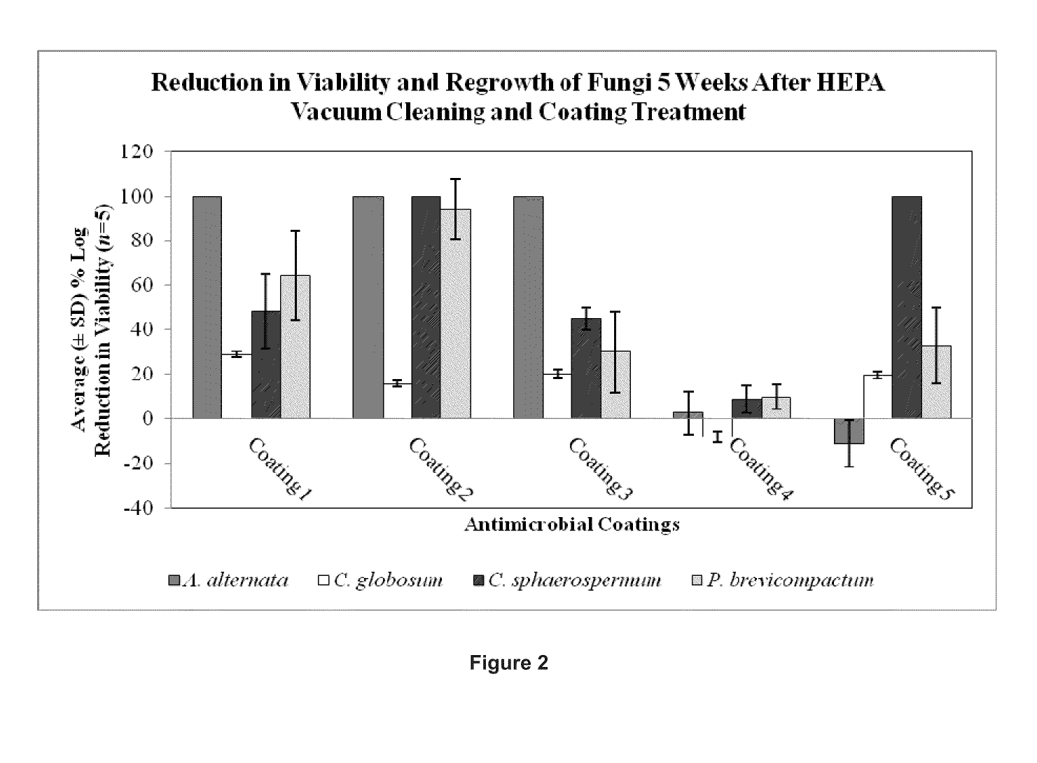

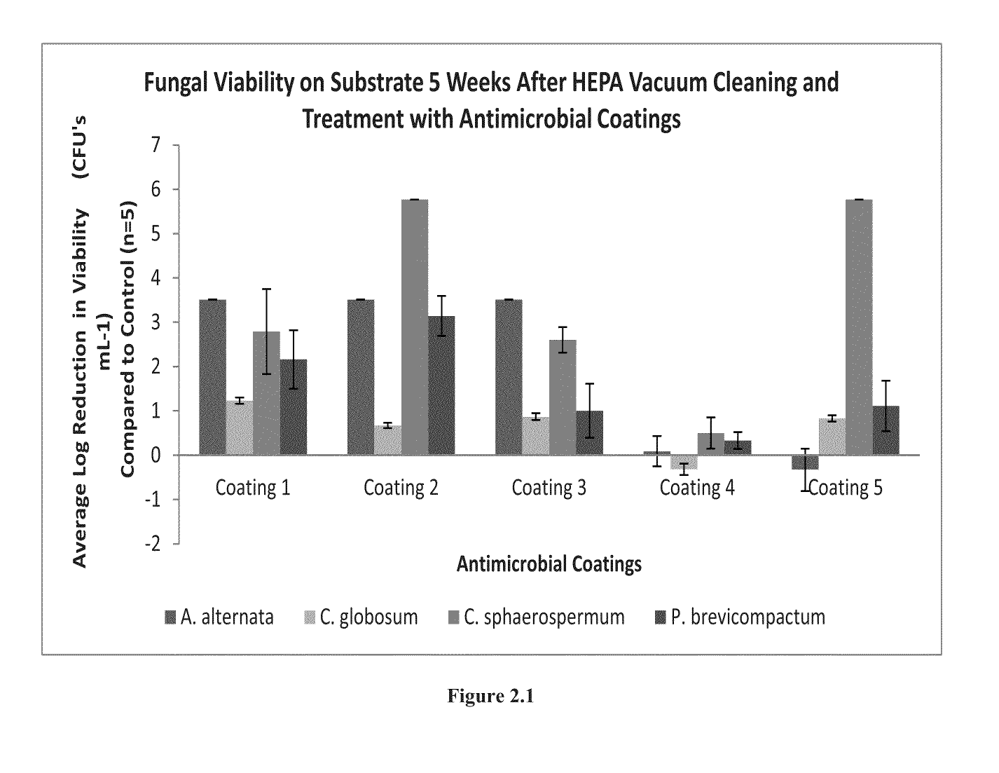

As illustrated in in FIGS. 2.1, 5.1, and 6.1, an alternative method of analysis involves determining whether or not the antimicrobial or antifungal agent is able to achieve a 3.0 or more log reduction in viability (CFU's mL-1) at the recommended “in-use” concentrations on contaminated surfaces, with or without prior cleaning. (see Disinfection, Sterilization, and Preservation, ed. S. Block, Lippincott, Williams & Wilkins, 2001, hereby incorporated by reference).

Further, log reductions were converted to percent (%) log reductions for comparison and statistical analysis. The comparison of average log reductions or average % log reduction values for multiple treatments was accomplished by the analysis of variance using one-way ANOVA and General Linear Modeling procedures using SPSS-17 (SPSS Inc. Statistical package for the social sciences, Version 17.0. Armonk, N.Y.) program, followed by Post Hoc analysis with Games-Howell test. The predetermined p-value for the acceptable level of statistical significance was <0.05. (t-Test is also applicable if comparing only two sets of data (i.e. two treatments)).

Results of HEPA Vacuum Cleaning Alone of Substantially Contaminated Substrates:

The residual viabilities of test fungi after contact cleaning of moldy substrates varied (Table 5). Log reductions in fungal viabilities were ≦1.0 in comparison to the negative control (not cleaned).

TABLE 5
Reduction in viability after contact cleaning of moldy surface
with an industrial strength vacuum cleaner (116.6 cubic feet minute-1
airflow) fitted with high efficiency particulate arrestor filter.

| | | Average log |
|---|---|---|
| Fungus | Carrier (n = 5). | reduction ± SD |
| Alternaria alternata | Gypsum wallboard | 0.64 ± 0.04 |
| Aspergillus versicolor | Gypsum wallboard | 0.53 ± 0.15 |
| Chaetomium globosum | Gypsum wallboard | 1.01 ± 0.05 |
| Cladosporium sphaerospermum | Gypsum wallboard | 0.20 ± 0.04 |
| Penicillium brevicompatum | Oriented strand board | 0.70 ± 0.04 |
| Penicillium brevicompactum | Plywood | 1.05 ± 0.40 |
| Stachybotrys chartarum | Gypsum wallboard | 0.92 ± 0.14 |

### Example 1

### Antimicrobial Coatings

Substrates with substantial microbial growth (fungal) that were subjected to HEPA vacuum cleaning followed by the application of antimicrobial coatings, (or treated as negative controls) did not show a change in appearance that was discernible to the unaided eye after 5 weeks. However, a determination of residual viability clearly established that fungal elements survived although to a variable degree (FIG. 2). Specifically: For A. alternate, residual viability was completely (100%) inhibited after 5 weeks by antimicrobial coatings 1, 2, and 3 on cleaned and coated substrates. In treatments with antimicrobial coatings 1 and 3, A. alternata differed significantly from the remaining fungi in this respect (p-value=<0.05). Alternaria alternate was resistant to antimicrobial coatings 4 and 5 post-cleaning. Further, only antimicrobial coating 1 marginally reduced the viability of C. globosum which showed lower susceptibility to the other formulations. Further, C. globosum was the only fungus that proved resistant to antimicrobial coating 2, differing significantly from other fungi (p-value=<0.05). Antimicrobial coatings 2 and 5 completely mitigated the viability of C. sphaerospermum whereas antimicrobial coatings 1 and 3 decreased the viability of C. sphaerospermum close to 50%. Antimicrobial coating 4 was ineffective against all test genera, Viability decreases were highest with coating 2, followed by coating 1 for P. brevicompatum. Antimicrobial coatings 3 and 5 were only marginally effective against this fungus. Antimicrobial coating 5 completely mitigated the viability of C. sphaerospermum alone, differing significantly from the viability reductions for remaining genera (p-value=<0.05), except A. alternate on which it had no effect.

### Discussion

Testing revealed that once fungal colonization has occurred, the reduction in fungal viability post-cleaning and post antimicrobial treatment varies with the antimicrobial formulation and target species. The absence of visible recurrence of vegetative or sporulating fungal mycelium on mechanically-cleaned and subsequently antimicrobial coated carriers for five weeks does not necessarily indicate elimination of viable residual fungal elements.

We demonstrate that cleaning with a high air-flow industrial vacuum cleaner to remove substantial growth results in the elimination of gross contamination of all fungi, but viable elements do survive that may amplify if conditions favorable for growth persist. Minor variations in cleaning efficiencies are expected depending on the characteristics of the material or substrate on which mold is growing, as noted for P. brevicompactum.

Variation in fungus susceptibility must be considered during the application of antimicrobial products. The test formulations display varied efficacy after the removal of mature growth. Antimicrobial coatings are not very effective against C. globosum post-cleaning. Nonetheless, C. globosum is one fungus which is quite efficiently removed from the carrier surface by contact vacuuming alone. It is much more difficult to inactivate C. globosum spores protected inside the residual perithecia with antimicrobial chemicals following cleaning. Antimicrobial coating 4 is ineffective against existing fungi. The other four antimicrobial coating products controlled the viabilities of one or more fungi when applied after HEPA vacuuming. The two best performing antimicrobial coatings with long lasting effects in the current investigation are multi-component formulations containing boron.

### Conclusion

The high airflow contact vacuuming removes superficial fungal growth from moldy surfaces but viable fungal elements remain, requiring additional remedial measures such as treatment with select antimicrobial coatings although outcome would ultimately depend on fungal and coating characteristics.

### Example 2

### Fungicides

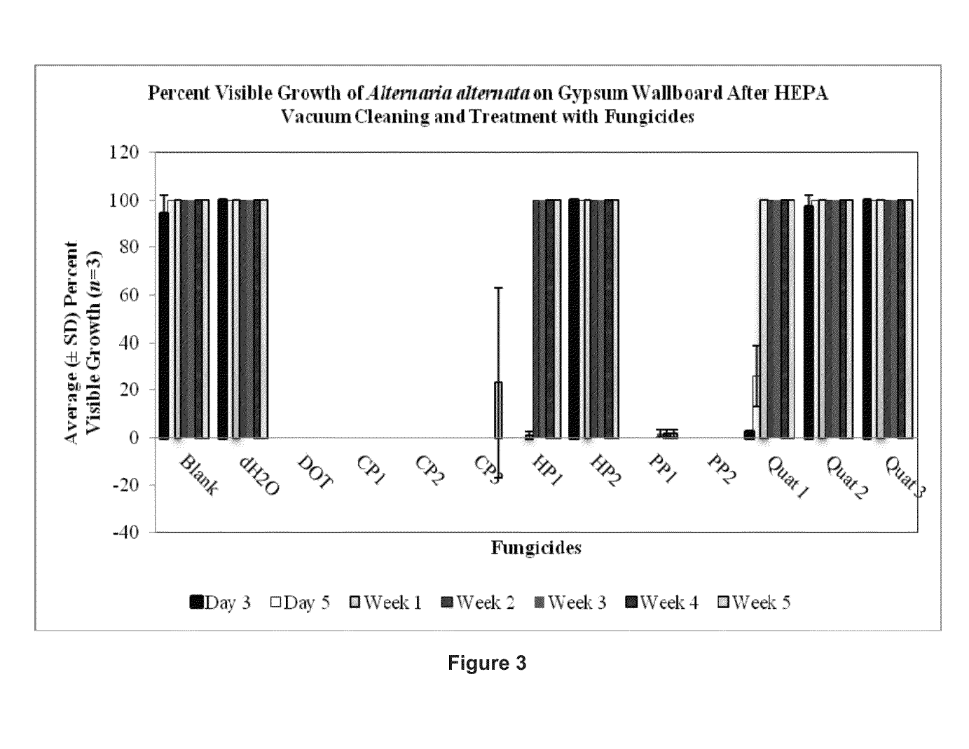

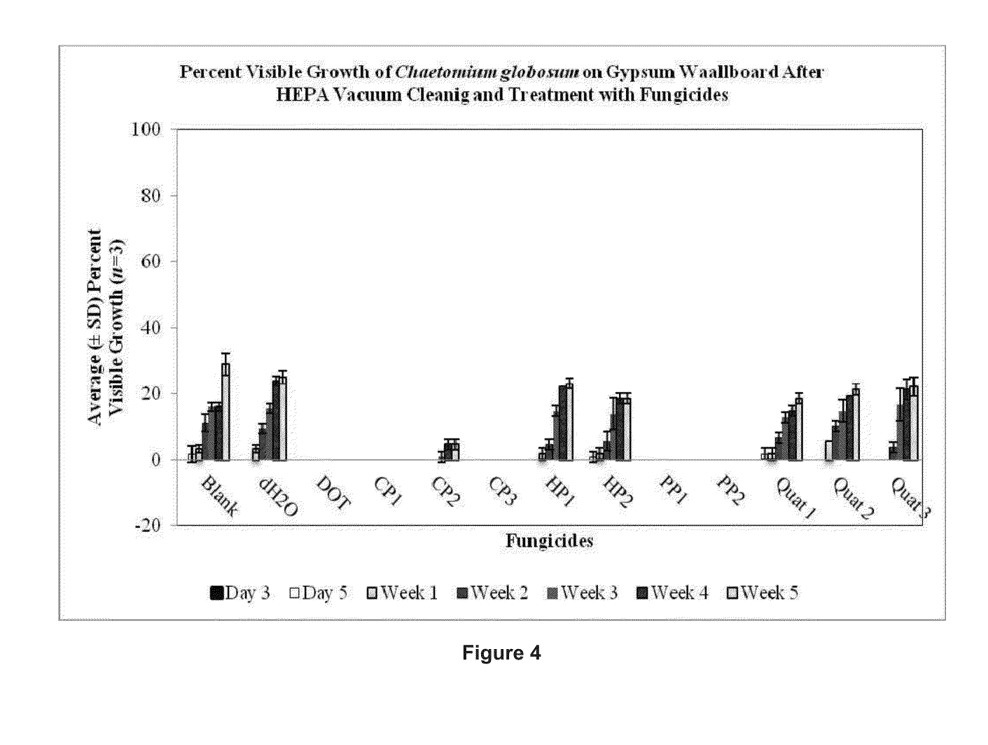

Recurrent growth of A. alternata and C. globosum on substrates 5 weeks after vacuum cleaning and fungicide treatment is shown in FIGS. 3 and 4. Results represent percent substrate surface area covered with visible fungal growth on the substrate surface. Compared to C. globosum, A. alternate grew very aggressively post-cleaning and chemical treatments. The recurrent growth of C. globosum by third week for HP1 and 2, Quats 1, 2, and 3 treatments did not vary significantly from that on the distilled water treated negative control coupons (p-value=>0.05), except that some delay in reemergence of growth with Quat 3 was noted. Quaternary ammonium products 1, 2, and 3, along with HP1 and 2 treatments resulted in 100% resumption of A. alternate growth by the end of week 1. Conversely, CP1, DOT, and PP2 treatments did not exhibit reemergence of growth of A. alternate and C. globosum 5 weeks post-treatment. Phenol Product 1 treatment completely prevented the recurrent growth of C. globosum in conjunction with minimal growth of A. alternanta. Chlorine Product 3 prevented growth of C. globosum, showing minimal and delayed recurrent growth of A. alternate. Conversely, minimal recurrent growth was observed for C. globosum with CP2 but none for A. alternate. The presence of vegetative or sporulating mycelium could not be discerned visually for P. brevicompactum and C. sphaerospermum on carrier coupons due to staining. Only residual viability data are presented for these two fungi.

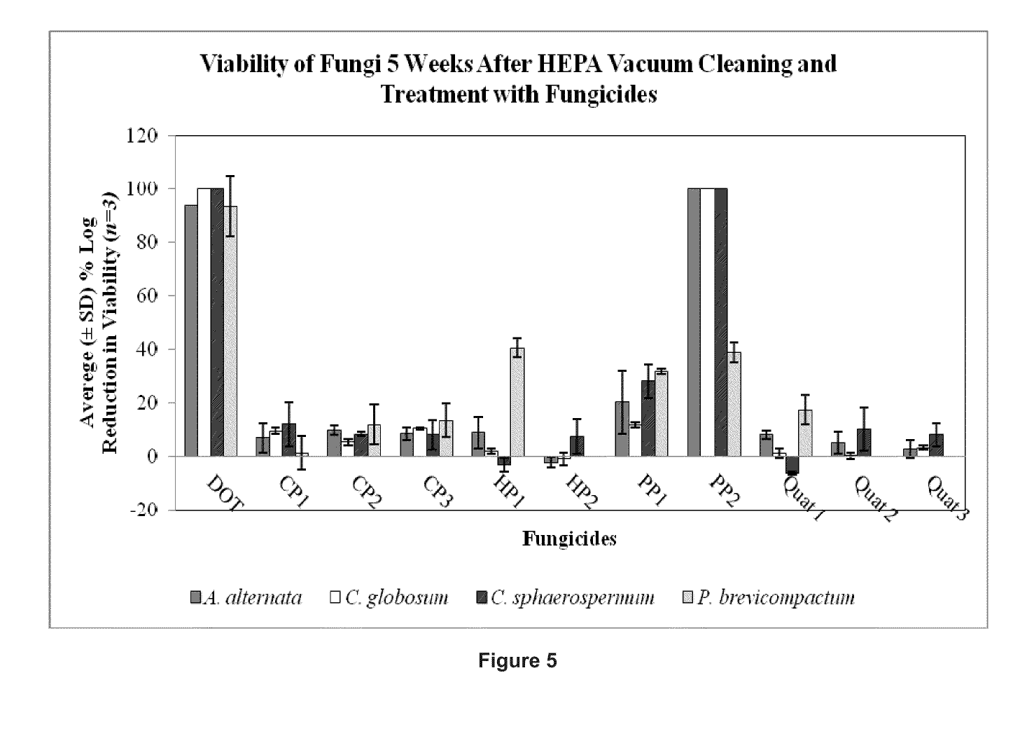

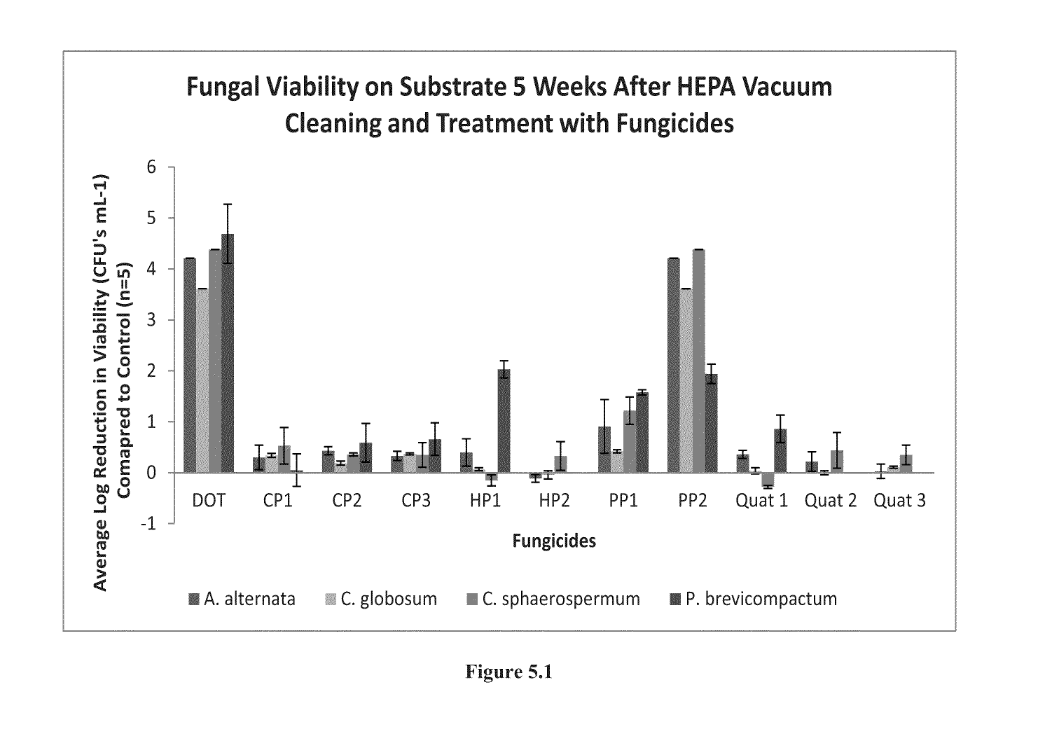

The determination of the residual viabilities showed that the absence of observable growth was not always indicative of the absence of viable fungal elements. Reduction in the viability of test fungi five-weeks following physical cleaning and chemical treatments are presented in FIG. 5. Phenol Product 2 and DOT were the two most effective products for controlling fungal viability long-term. Substrate and fungal-specificity were observed with PP2, with only 35% reduction in the viability of P. brevicompactum on OSB. The mean % log reductions in the viability of experimental genera did not vary significantly by fungi after treatments with CPs 1, 2, and 3, DOT, HP2, and Quats 2 and 3 (p-value=>0.05). Phenol Product 1 was effective against only C. sphaerospermum and P. brevicompactum. Significant differences (p-value=<0.05) were seen in the response of C. globosum and P. brevicompactum to PP1, and between A. alternate, C. globosum, and C. sphaerospermum with P. brevicompactum after treatment with PP2. The significance of the difference between C. globosum and C. sphaerospermum following treatment with PP1 was marginal (p-value=0.105) due to high variability in response of the later fungus. The reduction in residual viability subsequent to treatment with HP1 was highest for P. brevicompactum, with none or negligible effect on the remaining fungi (p-value=<0.05).

### Discussion

These results demonstrate that the disclosed assay, which utilized removal of gross contamination (5 week) and subsequent treatment with antimicrobial agents, provides more discriminating results and is therefore superior to other evaluation techniques, in particular suspension based testing (for examples of suspension testing see: AOAC Fungicidal Activity of Test Substances; and European standards, EN1275; and EN1650) Suspension may testing does not correctly predict biocidal efficacies under field use conditions where chemicals may be sprayed directly on the existing growth, or used after cleaning. Results from suspension testing methods reporting log reduction values may overestimate the reductions, as noted for bacteria, The inventors also discovered that the direct spraying of antimicrobial formulations on existing growth as performed in some existing methods (see AOAC Germicidal Spray Products Test) without prior mechanical removal of gross contaminations is less efficient in terms of fungicidal effect, then when fungal growth is removed followed by antimicrobial treatment.

Cleaning with an industrial strength vacuum cleaner alone reduces surface fungal growth but viable fungal elements survive (Table 5). Nonetheless, broad-spectrum, total, and long-term eliminations were possible after follow-up treatment with two of the eleven tested formulations (DOT and PP2). If conditions favorable for mold growth persist, then subsequent treatment of the cleaned moldy substrates with products containing boron or phenol compounds is essential for the prevention of reemergence of fungal growth, irrespective of the colonizing species.

Group 1 formulation (DOT) exhibits high and broad-spectrum effectiveness after 5 weeks of exposure. Total elimination of visible recurrent growth and residual viable elements is achieved suggesting that DOT is ideal for application after gross-contamination has been removed from smooth or rough surfaces as on the dry wallboard and OSB respectively, irrespective of the nature of colonizing fungus.

Group 2 chlorine releasing agents are comparatively less efficient than the Group 1 chemicals since residual viable elements remain even if the growth is not apparent post-cleaning for a minimum of 5 weeks. Further, the possibility of recurrent growth remains if favorable conditions continue or if rewetting of the budding materials occurs. The assay was able to reveal minor differences within this group. For example stabilized chlorine products (CP3 and 2) are not superior to sodium hypochlorite or household bleach in terms of overall fungicidal efficacy. In addition these results provide evidence that CP1 cannot be used for long-term protection due to residual fungal viability

Group 3 hydrogen peroxide (H₂O₂) based products are considered environmentally friendly. They remain active in the presence of organic load. Hydrogen Peroxide product 1 is marginally superior to HP2, showing delayed recurrent growth of A. alternata and C. globosum post-cleaning and spray treatment, but viable fungal elements remain.

Group 4 phenol based formulations generally provide excellent long-term protection when applied after mechanical cleaning. Our findings indicate that it works very well against all test fungi, including C. globosum, if applied post-cleaning for long-term growth control. The efficiency of PP1 is consistently lower than that of PP2, possibly due to difference in formulations.

Group 5 quaternary ammonium compound based formulations (Quat's) are surface-active cationic agents that may inhibit development of vegetative cell from germinated spore, but not the conversion of dormant to metabolically active state. The current results indicate that these formulations do not control recurrent growth post-cleaning. The efficacy ratings are among the lowest for the three quaternary ammonium compound based formulations with consistently mean viability log reduction values of <1.0 for test fungi when sprayed on surface growth after removal of the gross contamination from substrate coupons. Among the three formulations tested, only Quat 1 is generally recommended for use on surfaces of varied porosity. However, these results revealed that Quat 1 performed only marginally better on gypsum wallboard and OSB than Quat 3 or Quat 2.

Consistency and broad-spectrum efficacy are two highly desirable attributes of antimicrobial agents. The scientific literature supporting antifungal efficacies of many products tested in this study remains scant. In terms of the non-fungal-specific elimination of the residual viability by treatment after removing gross contamination, Group 1 and Group 4 products rate the highest. Both Group 3 and Group 5 rate the lowest for applicability in mold remediation. Some quaternary ammonium formulations may cause delay in recurrent growth but the effect is minor.

### Conclusion

Five-week carrier assay utilizing removal of fungal growth and subsequent treatment with antimicrobial chemicals is superior to the other disinfectant evaluation techniques. Only a small number of tested fungicides provide a broad-spectrum and long-term protection against fungi on building materials. Pre-cleaning of moldy surface is necessary to achieve long-term inhibition of fungal viability prior to the treatment with antimicrobial chemicals.

### Example 3

### Green Antimicrobial Agents

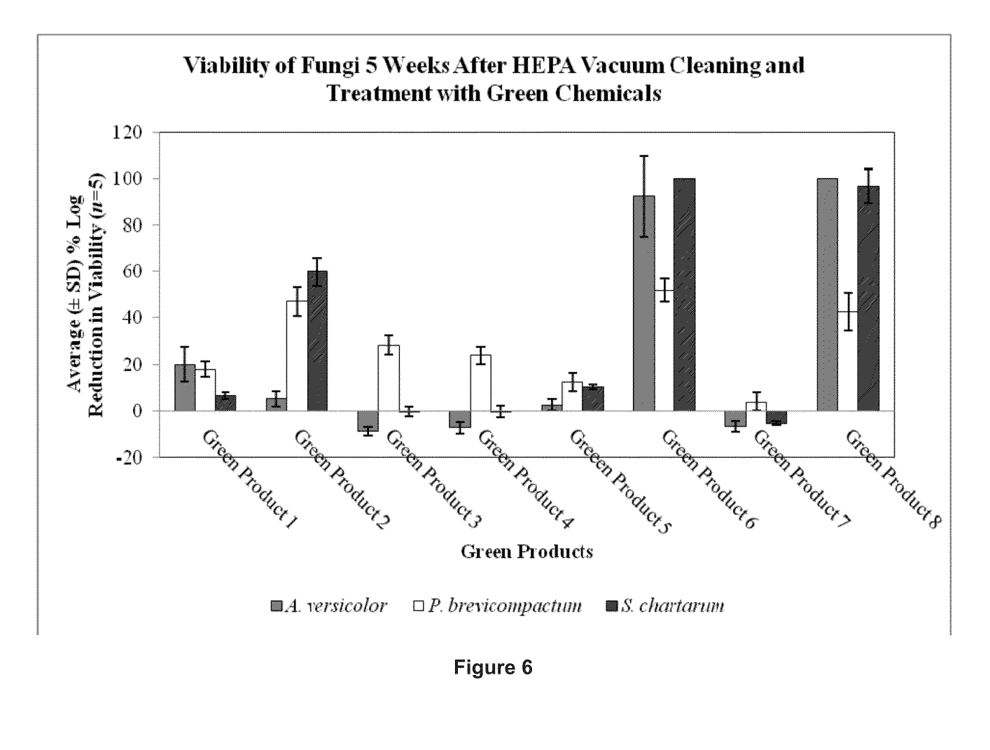

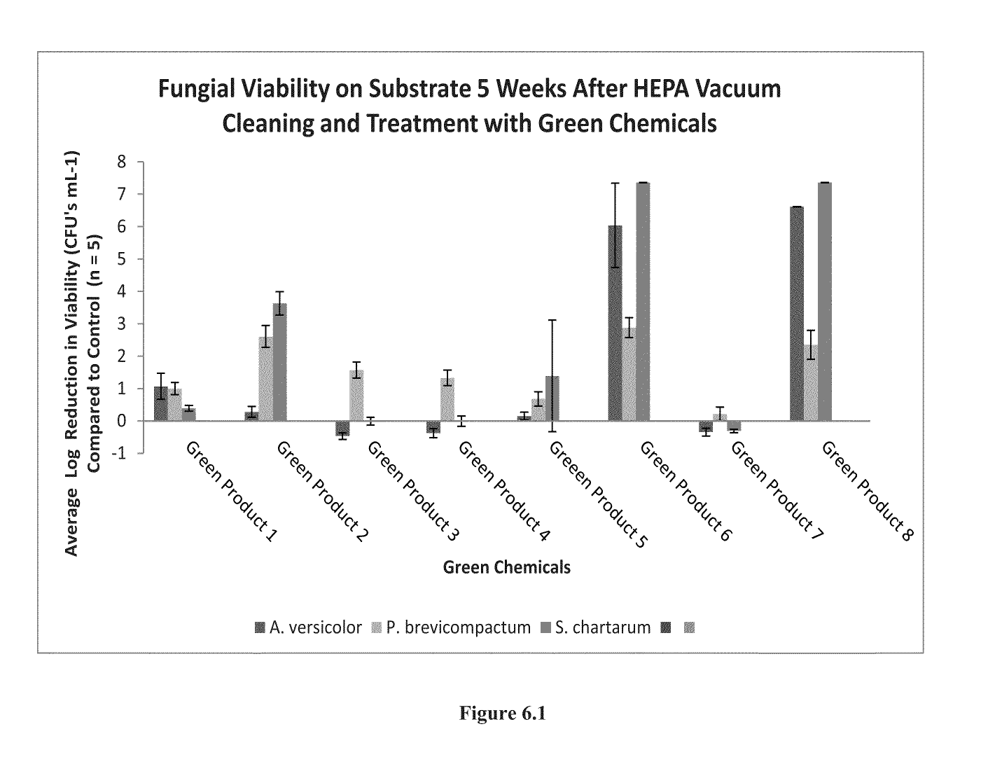

As reported above, the fungal re-growth on substrates with substantial microbial growth following HEPA vacuum cleaning and treatment with green antimicrobial agents could not be visually assessed due to residual staining or discoloration of the substrate surface. However, determination of the residual viabilities by extraction, 5 weeks after HEPA vacuum cleaning and treatment with green products clearly established that fungal elements survive, but to a variable degree (FIG. 6).

None of the eight test formulations completely reduced or mitigated the viabilities of all the three test genera (A. versicolor, P. brevicompactum, and S. chartarum), indicating selective efficacy. Post-cleaning treatments with only products 6 and 8 caused a nearly 100% or 100% average decrease in the viabilities of A. versicolor and S. chartarum, differing significantly from about 50% or less reduction for P. brevicompacium (p-value=<0.05). The next best formulation was product 2. It demonstrated 47% and 60% average decrease in the viability of P. brevicompactum and S. chartarum, respectively, followed by a minimal (5%) reducing effect on A. versicolor, The differences in response of three test fungi after treatment with this formulation were significant (p-value=<0.05). Conversely, green products 1, 3, 4, 5 and 7 caused negligible or marginal reductions in the viabilities of the experimental fungi. Penicillium brevicompactum differed significantly from A. versicolor and S. chartarum following treatment with products 3 and 4, showing higher but borderline reducing effect compared to none or negligible for the other genera (p-value=<0.05).

### Conclusion

Not all green products are 100% effective against Aspergillus versicolor and Stachybotrys chartarum in treatment after removing the superficial growth from moldy building material surface. The viability of Penicillium brevicompactum regardless of wider susceptibility, is reduced at most about 50%. Therefore, fungicidal effectiveness varies with the green product formulation, colonizing species and possibly with substrate.

The discussion of the references herein is intended merely to summarize the assertions made by the authors and no admission is made that any reference constitutes prior art. Applicants reserve the right to challenge the accuracy and pertinence of the cited references.

1. A method of determining the effectiveness of an antimicrobial agent in reducing or inhibiting recurrent microbial growth, the method comprising:
a) obtaining a substrate with visible substantial microbial growth or gross contamination,
b) removing the visible substantial microbial growth or gross contamination from the substrate by mechanical cleaning,
c) treating the substrate with the antimicrobial agent,
d) incubating the substrate for one or more periods of time at a temperature and relative humidity that will allow growth of the microbe,
e) determining viable colony forming units by extracting viable microbes from the substrate and plating the extract on nutritive media, then incubating the viable microbes on the nutritive media in an environment that allows growth of the viable microbes and,
f) comparing the viable colony forming units obtained from the antimicrobial treated substrates, to viable colony forming units obtained from substrates not treated with an antimicrobial, or to viable colony forming units obtained from substrates treated with different antimicrobials and subjected to steps a) through e).

2. The method of claim 1, wherein incubating the substrate for one or more periods of time to determine efficacy of the antimicrobial, comprises incubating the substrate for a period of time determined to represent short term efficacy.

3. The method of claim 2, wherein the time determined to represent short term efficacy consists of about 24 hours.

4. The method of claim 1, wherein incubating the substrate for one or more periods of time to determine efficacy of the antimicrobial, comprises incubating the substrate for a period of time determined to represent long term efficacy.

5. The method of claim 4, wherein the time determined to represent long term efficacy is about 5 weeks.

6. The method of claim 1, wherein the substrate with substantial microbial growth is obtained from the field.

7. The method of claim 1, wherein the substrate with substantial microbial growth is cultivated in the laboratory.

8. The method of claim 1, wherein the substantially contaminated substrate is mechanically cleaned using high airflow vacuum.

9. The method of claim 1, further comprising an assessment of substrate surface growth.

10. A method of determining the effectiveness of an antimicrobial agent in reducing or inhibiting microbial growth on a substantially contaminated substrate, the method comprising:
a) obtaining a substrate with substantial microbial growth or gross contamination,
b) mechanically cleaning the substrate to remove with substantial microbial growth or gross contamination,
c) treating a substrate with the antimicrobial agent,
d) incubating the substrate for one or more periods of time to determine efficacy of the antimicrobial, at a temperature and relative humidity that will allow growth of the microbe,
e) assessing substrate surface growth,
f) determining viable colony forming units by extracting viable microbes from the substrate and plating the extract on appropriate nutritive media and under appropriate environmental conditions that would allow growth of the microbe and,
g) comparing the viable colony forming units and the assessed substrate surface growth, obtained from the antimicrobial treated substrates to viable colony forming units and assessed substrate surface growth obtained from substrates not treated with an antimicrobial or to viable colony forming units obtained from substrates treated with different antimicrobials and subjected to steps a) through f).

11. The method of claim 10, wherein incubating the substrate for one or more periods of time to determine efficacy of the antimicrobial, comprises incubating the substrate for a period of time determined to represent short term efficacy.

12. The method of claim 11, wherein the time determined to represent short term efficacy consists of about 24 hours.

13. The method of claim 10, wherein incubating the substrate for one or more periods of time to determine efficacy of the antimicrobial, comprises incubating the substrate for a period of time determined to represent long term efficacy.

14. The method of claim 13, wherein the time determined to represent long term efficacy is about 5 weeks.

15. The method of claim 10 wherein the substrate with substantial microbial growth is obtained from the field.

16. The method of claim 10, wherein the substrate with substantial microbial growth is cultivated in the laboratory.

17. The method of claim 10, wherein the substantially contaminated substrate is mechanically cleaned using high airflow vacuum.

18. The method of claim 10, further comprising an assessment of substrate surface growth.
